(19) United States
(12) Patent Application Publication
(10) Pub. No.: US 2007/0196900 A1
(43) Pub. Date: Aug. 23, 2007

(54) METHOD FOR THE MANUFACTURE OF LANTIBIOTICS

(76) Inventors: Hubert Muellner, Kelkheim (DE); Martin Folger, Ingelheim (DE); Andreas Werner, Mainz (DE); Uwe Gierlich, Mainz (DE); Kurt Eyer, Glis (CH); Klaus Heinzmann, Visperterminen (CH); Nicholas Shaw, Visp (CH); Fabian Wyer, Lalden (CH)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/674,664

(22) Filed: Feb. 13, 2007

(30) Foreign Application Priority Data
Feb. 13, 2006 (EP) 06101571

(51) Int. Cl. C12P 21/06; C12N 1/21; C07K 7/60

(52) U.S. Cl. 435/69.1; 435/252.3; 435/471; 530/317

(57) ABSTRACT

The present invention relates to the manufacture of antibiotic compounds of the class known as lantibiotics. Preferably, the present invention relates to the purification of those lantibiotics.

### BACKGROUND OF THE INVENTION

1. Field of the Invention

This application claims priority of EP 06101571, filed on Feb. 13, 2006, which application is incorporated herein by reference in its entirety.

The present invention relates to the manufacture of biomolecules. In particular, the present invention relates to the manufacture of antibiotic compounds of the class known as lantibiotics. Preferably, the present invention relates to the purification of those lantibiotics.

2. Description of the Prior Art

Lantibiotics are a class of small peptide antibiotics characterized by the presence of unusual, bridged thioether amino acids, namely lanthione and 3-methyllanthione. Members of this class include subtilin, nisin, epidermin, gallidermin (which is the leu-6 variant of epidermin), pep5, ancovenin, Ro 09-0198, cinnamycin and duramycin. Pep5, Epidermin and Gallidermin are all naturally produced by microorganisms of the genus Staphylococcus.

Fermentation of the Gallidermin-producing strain S. gallinarium Tü3928 (DSM4616) is described in detail in EP-342 486, Kellner et al., Eur. J. Biochem. 177:53-59 (1988), Hörner et al., Appl. Microbiol. Biotech. 30, 219-225, and in Ungermann et al., 1^{st} Proc. Int. Workshop Lantibiotics, Tübingen 1991, p. 410-421. Briefly, the media used for the fermentation of Gallidermin comprises at least meat extract, calcium chloride and sodium chloride. The feeding solution comprises meat extract and glucose. Thus, the media used for the fermentation of lantibiotics are very complex, and have large amounts of oligo or polypeptides as the amino acid source.

EP-508 371 A describes a purification strategy for lantibiotics based on a chromatographic procedure. The process comprises multiple chromatographic steps, that are time, material and cost consuming. Briefly, EP 508 371 provides a process for lantibiotic purification comprising obtaining a lantibiotic containing fermentation medium and subjecting said medium or lantibiotic containing media deriving therefrom to successive steps of adsorption on a styrene divinyl copolymerizate matrix, cation exchange chromatography (e.g. Amberlite XAD-1180®), hydrophobic interaction chromatography, optionally but preferably anion exchange chromatography, desalting by ultrafiltration and/or diafiltration and, optionally, lyophilisation. Extra purification steps can of course be used if desired. Yields of about 50% were achieved by the disclosed purification procedure. The fed-batch process described therein lead to volumetric yields of 840 mg/L.

US 2004/0072333 A1 describes a proteolytic purification method for lantibiotics, using nisin as an example. Careful, fine tuned protease treatment leaving nisin unaffected is used to eliminate contaminating peptides that are difficult to remove from crudely purified product.

In summary, the production processes for lantibiotics known in the art are very complex and require several purification steps. Thus there is a need for a simple and cheap production procedure for lantibiotics in the art.

### BRIEF DESCRIPTION OF THE DRAWINGS

FIG. 1 shows detailed feeding-strategies for the process as described herein.

FIG. 2 shows the typical profiles of two standard fermentations resulting in a productivity of gallidermin as described herein.

### SUMMARY OF THE INVENTION

The present invention relates to a simple, time-saving production process for lantibiotics. The production process described herein, is based on a new fermentation concept, with less complex media as compared to those described in the art, and a simple one or two step purification procedure, comprising an initial precipitation step with an inorganic salt. Thus, the present invention relates to a method for the manufacture of a lantibiotic peptide, comprising a fermentation and purification step, wherein the purification step comprises the step (i) precipitation of the lantibiotic peptide from a cell culture supernatant by adding an inorganic salt. According to more preferred embodiment, the purification procedure further comprises the purification step: (ii) subjecting the peptide obtained from the precipitate of step (i) to a washing step, a single chromatographic purification step, a drying step or to crystallization. According to a further more preferred embodiment, the fermentation is performed in a medium comprising maltose, calcium chloride, and hydrolyzed yeast extract with a high amount of free amino acids and simple oligopeptides, preferably more than 50% of the proteineous components of said yeast extract are free amino acids. More preferably, the fermentation medium does not include any meat or peptone extract.

### DETAILED DESCRIPTION OF THE INVENTION

Before the embodiments of the present invention it must be noted that as used herein and in the appended claims, the singular forms “a”, “an”, and “the” include plural reference unless the context clearly dictates otherwise. Thus, for example, reference to “a lantibiotic” includes a plurality of such lantibiotics, reference to the “bacterium” is a reference to one or more bacterium/bacteria and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, genetic material, and methodologies as reported in the publications which might be used in connection with the invention. Nothing herein is to be construed as an admission that the invention is not entitled to antedate such disclosure by virtue of prior invention. Moreover, all processes described herein and are not known to the public, are considered to be a process according to the invention.

The present invention related to a process for the production of lantibiotics. Lantibiotics are peptide antibiotics containing lanthionine. Typically, lantibiotics are polycyclic polypeptide antibiotics with a high content of unsaturated amino acids (dehydroalanine, dehydrobutyrine) and thioether amino acids (meso-lanthionine, (2S,3S,6R)-3-methyllanthionine). Furthermore, lysinoalanine, 3-hydroxyaspartic acid and S-(2-aminovinyl)-D-cysteine have been found in some members of the lantibiotics. Exemplarily, the following lantibiotics are known in the art: nisin, subtilin, duramycin, cinnamycin, ancovenin epidermin, Ro09-0198, pep5, lacticin 481 and 3147, mersacidin, actagardin, mutacin 1140, gallidermin. A summary of these lantibiotics is found for example in Kellner et al, (supra) or in Current Protein and Peptide Science 2005, no. 6, pp. 61-75 (Cotter at al.,). The production process described herein preferably is applicable to the production of epidermin and gallidermin, more preferably to the production of gallidermin and most preferred to gallidermin comprising at least the structural motif of formula I:

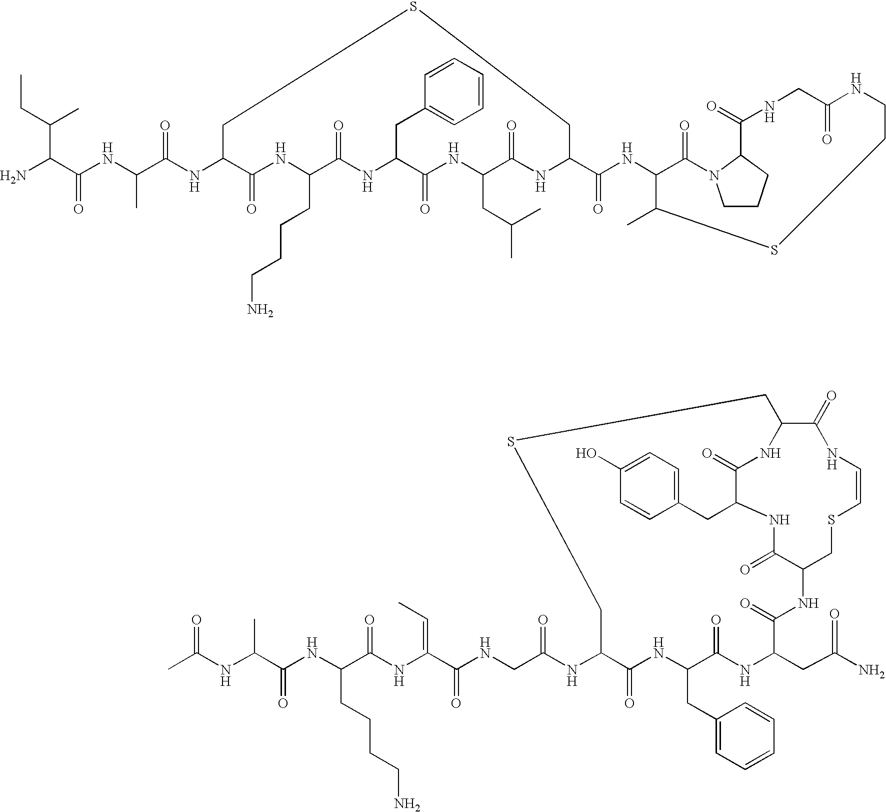

It is hereby understood that the process for the manufacture of the lantibiotic, as described herein, comprises a fermentation step and a purification step. For clarity, both steps are separately described more in detail. However, each embodiment described for the fermentation process can be combined with any embodiment described for the purification step in order come to a final manufacture process.

The Fermentation Process:

The general understanding existing in the prior art for an optimized production process for lantibiotics relates to a process with a maximal amount in total of lantibiotics within the fermentation step. As a consequence, nutrient-rich and complex media, comprising meat or peptone extracts were used for the fermentation process. In contrast thereto, the present invention is based on the finding that a more controlled fermentation process of the lantibiotics, preferably of gallidermin, facilitates the purification of the lantibiotic peptides and therefore results in a more efficient and economical production process. It was surprisingly found that the fermentation process described herein allows the purification of lantibiotics from the culture supernatant with a “high degree of purity” by a simple one or two step purification strategy comprising an initial precipitation step with an inorganic salt.

“A high degree of purity” according to the present invention means, a product purity of at least 90% (w/w), preferably of at least 92%, more preferred of at least 94%, furthermore preferred of at least 96% furthermore preferred of at least 98% furthermore preferred of at least 99% with respect to the drug product.

Lantibiotics can be easily produced in gram-(+) bacteria known to the state of the art. For example, epidermin and galidermin can be easily produced in Staphylococcus spp. coding for and expressing the relevant genes for epidermin and/or gallidermin production. It is described in the prior art that gallidermin, for example, can be efficiently produced in Staphylococcus gallinarium strain Tü3928. This strain is described in detail in EP A-342 486. It has been deposited by the Deutsche Sammlung für Mikroorganismen und Zellkulturen (DSMZ) in Braunschweig, Germany under the accession no. 4616. Thus, any bacterial strain known in the art can be used for the production of the lantibiotics as described herein. For the production of gallidermin as described herein, use of Staphylococcus gallinarium strain Tü3928 (DSM 4616) is most preferred.

The fermentation of the microorganisms capable of producing lantibiotics is known in the art. The microorganism can be fermented in liquid medium after inoculation with a suitable inoculum under any conditions suitable for that particular microorganism, either in batch, fed-batch or continuous mode. Microorganisms of the genus Staphylococcus can be fermented under aerobic conditions, preferably at a temperature between 24 to 37° C. and more preferably at a pH between about 5.6 and 8.5, preferably between about 6.0 and 8.0, most preferred at about pH 7.3.

Suitable basal culture media for the fermentation of lantibiotics, preferably gallidermin, contain maltose, a calcium source, and yeast extract. Preferably, those basal culture media may comprise additive(s) that prevent foam formation during the fermentation process. More preferred those media do not include any meat or peptone extract. It was surprisingly found, that meat/peptone extracts used in the art contain a high amount of proteineous components that negatively affect the production process of lantibiotics, preferably of gallidermin. Thus, according to another embodiment, the culture media provided herewith, consist of demineralized water, maltose, a calcium source, yeast extract and at least one anti-foaming agent.

As a calcium source, CaCl₂ is preferably used. More preferably, CaCl₂ is used at an amount of about 10 to 200 mg/L culture medium, even more preferred of about 10 to 100 mg/L, even more preferred of about 20 to 80 mg/L, even more preferred of about 30 to 60 mg/L, even more preferred of about 40 to 50 mg/L, most preferably at about 42 to 48 mg/L. However, it is also in the knowledge of a person skilled in the art to substitute, or at least partially substitute the amount of CaCl₂ within the basal medium by other suitable calcium sources in that Ca²⁺ is available in equivalent concentrations in the medium (about 90 μM to 1.8 mM). Moreover, it is also in the knowledge of a person skilled in the art to reduce the amount of the CaCl₂, or any equivalent calcium source in the basal medium and to continuously or discontinuously feed it during the fermentation process.

The maltose, preferably used as maltose monohydrate, is preferably added to the basal medium in amounts of about 0.5 to 20 g/L medium (corresponds to about 1.39 to 55.5 mM of maltose monohydrate). More preferably, the amount of maltose in the basal medium is between about 1 to 5 g/L, even more preferred between about 2.5 to 7.5 g/L, most preferred about 5 g/L. However, it is also in knowledge of the person skilled in the art to substitute or at least partially substitute the maltose by any metabolic precursor of maltose that can be also consumed by the producer, or any equivalent carbon source. However, for the initial growth phase of the fermentation process, the use of “indirect carbon sources” is preferred. Indirect carbon sources, are for example di-, oligo-, or polysaccharides, such as maltose, but not glucose. Moreover, it is also in the knowledge of a person skilled in the art to reduce the amount of maltose, any metabolic precursor or suitable equivalent in the basal medium and to continuously or discontinuously feed it during the fermentation process.

It was surprisingly found, that the origin and quality of the protein source seems to be critical for production process of lantibiotics, at least in respect to a combined fermentation and purification process, as described herein. In contrast to fermentation processes described in the prior art—all of them make use of peptone or meat extracts—, the medium used for the fermentation of lantibiotics described herein, comprises yeast extract as preferred, preferably as sole protein and amino acid source. However, addition of trace amounts does not negatively affect the production process of lantibiotics as described herein.

Preferably, the yeast extract is added to the basal medium in an amount of about 10 to 200 mg/L culture medium, even more preferred of about 10 to 100 mg/L, even more preferred of about 20 to 80 mg/L, even more preferred of about 30 to 70 mg/L, even more preferred of about 40 to 60 mg/L, most preferably at about 50 mg/L. However, it is also in the knowledge of a person skilled in the art to reduce the amount of the yeast extract in the basal medium and to continuously or discontinuously feed it during the fermentation process.

Preferably, more than about 50% in total of the amino acids of the yeast extract are free amino acids and/or di-peptides. More preferably more than about 55%, even more preferred more than about 60%, even more preferred more than about 65%, even more preferred more than about 70%, even more preferred more than about 75%, even more preferred more than about 80%, even more preferred more than about 85%, and most preferred more than about 90% in total of the amino acids of the yeast extract are free amino-acids or di-peptides.

According to another embodiment, more than about 50%, preferably more than about 55%, more preferred more than about 60%, even more preferred more than about 65%, even more preferred more than about 70%, even more preferred more than about 75%, even more preferred more than about 80%, even more preferred more than about 85%, and most preferred more than about 90% of the amino acids Asp, Glu, Asn, Gly, Ser, Thr, Ala, and Arg of the yeast extract used for the fermentation of the lantibiotics, e.g., gallidermin, as described herein, are free-amino acids or exist in forms of di-peptides.

The basal media furthermore may comprise anti-foam agents that prevent or reduce foam formation during the fermentation process. Those “anti-foam agents” may be added in suitable amounts to the basal media, known to a person skilled in the art. For example, anti-foam agents are ionic or non-ionic surfactants, such as pluronic acids, polyethylene glycols, potyvinyl-pyrrolidone (PVP), polyvinyl alcohol (PVA), alcoholic EO/PO adducts, such as Genapol EP, etc. Preferably, a mixture of polyethylene glycol 600 and Genapol® EP00244 (Clariant, Germany) is used.

The fermentation process is preferably performed between about 24 and 37° C., preferably between about 28 to 37° C., even more preferred between about 32 and 37° C., even more preferred between about 35and 37° C., most preferred about 37° C.

The pH value is adjusted to about pH 5.6 to 8.0. According to more preferred mode, the pH values at start of fermentation is adjusted about pH 5.6 to 8.0, more preferably to about pH 6.5 to 7.5, even more preferred to about 6.8 to 7.5, most preferred to about 7.0 to 7.5.

The fermentation process is preferably performed in a fed-batch mode. Fed-batch processes for the fermentation of bacteria are well known in the art to a skilled person. Briefly, a bio-fermenter is filled with a basal medium and inoculated with bacteria, preferably with 2.0-20 mL (OD₆₀₀=10) of a bacterial culture/L fermenter broth. During the fermentation process, a feed mix, comprising suitable nutrients, is discontinuously or continuously added. Thus, according to a further embodiment, the fermentation process for the production of lantibiotics, as described herein, is performed in a fed-batch mode.

Preferably the feed-mix used for the fermentation process as described herein, comprises at least one energy source and further nutrients which are preferably consumed by the producer during the fermentation process. For example, the feed-mix may comprises a carbon source, preferably selected from the group sugars, sugar alcohols, amino sugars, uronic acids, amino acids, glycerine, glycerol ester. According to a further embodiment of the process as described herein, the feed mix comprises one or more monosaccharides, e.g. glucose, fructose, mannose, galactose, etc, or a mixture thereof. According to a further embodiment of the process as described herein, the feed-mix comprises a sugar, preferably a monosaccharide, and one or more amino acids, preferably selected from the group of Asp, Glu, Asn, Gly, Ser, Thr, Ala and/or Arg. According to a further embodiment of the process as described herein, the feed-mix comprises glucose or consisting of glucose.

There are several feeding strategies known in the prior art, that can be applied to the production of lantibiotics, preferably gallidermin, in a fed-batch mode. Preferably, the feeding starts, after the pO₂ within the medium is shifted below to 80 mbar, preferably below to 60 mbar, even more preferred below to 40 mbar. Preferably the feeding rate is increased to about 3-10 kg/(m³ h), more preferably about 4-8 kg/(m³ h), even more preferably about 5-7 kg/(m³ h), most preferably about 6.5 kg/(m³ h). According to a further embodiment of the fermentation process described herein, the basal feeding rate may be continuously and/or stepwise increased during the fermentation. For example, the continuously increasing rate may be to about 0.01-0.5 kg/(m³ h), preferably to about 0.05-0.3 kg/(m³ h), more preferably to about 0.1-0.25 kg/(m³ h), even more preferably to about 0.15 kg/(m³ h).

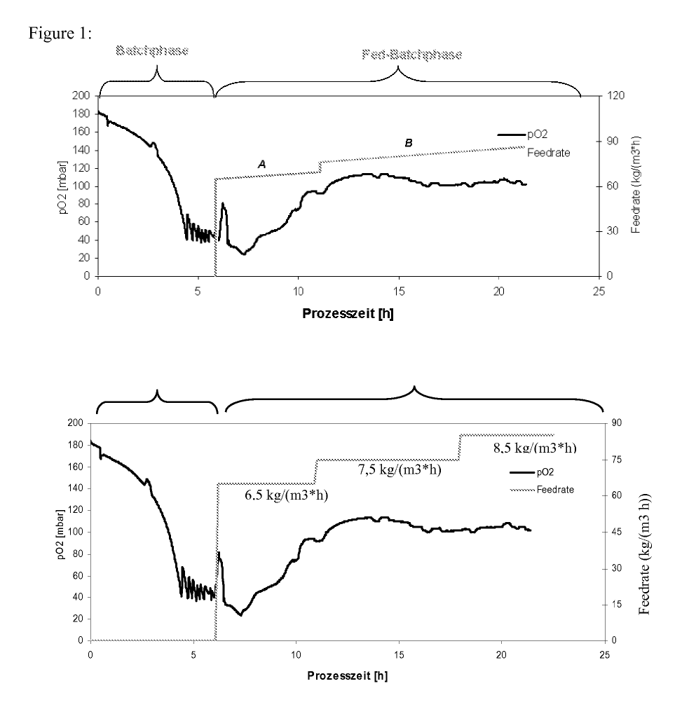

Further, more detailed feeding-strategies for the process as described herein are disclosed in FIG. 1. Briefly, after the pO₂ within the medium is shifted below to 80 mbar, preferably below to 60 mbar, even more preferred below to 40 mbar, a feed mix is added with an initial rate of about 3-10 kg/(m³ h), more preferably of about 4-8 kg/(m³ h), even more preferably of about 5-7 kg/(m³ h), most preferably of about 6.5 kg/(m³ h). The initial feeding rate is continuously increased with an increasing rate of about 0.01-0.5 kg/(m³ h), preferably of about 0.05-0.3 kg/(m³ h), more preferably of about 0.1-0.25 kg/(m³ h), even more preferably of about 0.15 kg/(m³ h). Preferably, after the pO₂ is returned to about 100 mbar, the feeding rate (exists at that time) is once increased for about 1-2 kg/(m³ h), preferably for about 1 kg/(m³ h).

Alternatively, after pO₂ within the medium is shifted below to 80 mbar, preferably below to 60 mbar, even more preferred below to 40 mbar, a feed mix is added with an initial rate of about 3-10 kg/(m³ h), more preferably of about 4-8 kg/(m³ h), even more preferably of about 5-7 kg/(m³ h), most preferably of about 6.5 kg/(m³ h). The initial feeding rate is increased over the fermentation process in two steps, each step for about 0.5-2 kg/(m³ h), preferably for about 1 kg/(m³ h).

The termination of the formation seems to be important for the overall production process as described herein. It has been surprisingly found, that when the fermentation process is terminated before the onset of the pseudo stationary phase, a single purification process as described herein can be applied, resulting in highly purified lantibiotics, e.g., gallidermin. In other words, for the production of lantibiotics according to the process as described herein, the cell culture is grown to no longer than to the onset of stationary phase. By term “no longer than the onset of the stationary phase” a fermentation process is meant that is stopped before the pH is <5.6, preferably at 6.0. Alternatively, the term “no longer than the onset of the stationary phase” means a fermentation process, in that total number of viable cells within the fermenter is not increased further. Moreover, the term “no longer than the onset of the stationary phase” also means that the fermentation is stopped before the pH is <6.0 and the total number of viable cells is not longer increased. The total number of cells can be estimated directly or by use of indirect methods, such as for example by estimation of the turbidity of the culture broth. Normally, the turbidy of a cell culture increases when the number of cells increases. “Turbidity”, for example can be estimated as optical density (O.D.₆₀₀) measured at a wavelength of 600 nm, per ml culture broth and time. Thus, the term “no longer than the onset of the stationary phase” also means, that the value for ΔO.D.₆₀₀ per ml fermenter broth is dropped down to less than 0.1 for 10 min. fermentation. Preferably the value for ΔO.D.₆₀₀ per ml fermenter broth and 10 min. fermentation time is dropped down to less than 0.05, more preferably to less than 0.01. Thus, alternatively the term “no longer than the onset of the stationary phase” also means that the fermentation is stopped before the pH is <5.6, preferably <6.0 and/or that the value for ΔO.D.₆₀₀ per ml fermenter broth is dropped down to less than 0.1 for 10 min. fermentation. Preferably the value for ΔO.D.₆₀₀ per ml fermenter broth and 10 min. fermentation time is dropped down to less than 0.05, more preferably to more than 0.01.

The Purification Process:

It was the common understanding that for increased production of the lantibiotics e.g. epidermin and gallidermin, it is important to remove those products during the fermentation process, because lantbiotics are harmful to the producer (bacterium) itself, and are subject to the activity of proteases excreted from the producing strains. As a result, dialyse or discontinuous adsorption chromatographic steps are integrated into the fermentation process (see, e.g., Ungermann et al, (supra)), that allow a continuous separation of the lantibiotic from the culture broth. The fermentation process as described herein does not need such a process, even if such a separation step can be used according to a further embodiment of production process as described herein.

It has surprisingly found that lantibiotic peptides, in particular gallidermin, can be purified at high yields and purity from the culture broth (culture supernatant) by an initial salt precipitation step. The term “initial salt precipitation step” means that the culture broth is not subjected to any other purification steps prior to the salt precipitation. However, cell separation or adjustment of the pH are not considered as purification steps.

Thus, the present invention also relates to a method for the manufacture of a lantibiotic peptide, preferably gallidermin, comprising fermentation and a purification step, wherein the purification step comprises the step (i) precipitating a lantibiotic peptide from a cell culture supernatant by adding a salt, preferably an inorganic salt. It has been surprisingly found that lantibiotics show unexpected precipitation properties, when subjected to salts, preferably to inorganic salts. Preferably, those precipitation properties have been shown in combination with the fermentation process as described herein (supra). In other words, the efficacy of the salt precipitation step can be increased, when the lantibiotics are fermented according to a method as described herein. For example, the precipitation experiments with alkaline halogen salts or ammonium sulphate salts resulted in a product yield of about 80 to 90% in total in combination with purities of about 90 to 94% in total.

Salt precipitation procedures—or salting out processes—are well known to a person skilled in the art, and were often used in combination with further purification steps. For example salting out is described in Harris and Angal (eds.) in protein purification methods—a practical approach, Oxford University Press 1995. However, it has never been described, that a single precipitation step results in such a high product purity and yield. Suitable salts for carrying out the precipitation step as described herein are inorganic salts.

Several aspects of the salts used should be considered. The effectiveness of the salt is mainly determined by the nature of the anion, multi-charged anions being the most effective. The order of effectiveness is phosphate>sulphate>acetate>chloride>(and followed by the Hofmeister series). Although phosphate is more effective than sulphate, in practice phosphate consists of mainly HPO₄²⁻ or H₂PO₄⁻ at neutral pH, rather than the more effective PO4³⁻. Monovalent cations are most effective, with NH⁴>K¹>N¹. The solubility is also an important consideration, since concentrations of up to several molar are required. Thus, many potassium salts are not suitable in respect to this aspect. Because of the risk of possible denaturation, or changes in solubility, there should be little increase in heat caused by salt dissolving. The final consideration is the density of the resultant solution, since the difference between the densities of aggregates and the solution determines the ease of separation by centrifugation.

Preferably, salts of the formula: MⁿX^{m}, wherein n=+1, +2 and m=−1,−2,−3 are used for the precipitation of the lantibiotics according to the process as described herein. Even more preferred are inorganic salts, characterized in that X is selected from the group comprising halogen, phosphate, phosphonate, sulphate, sulphonyl, acetate and in that M is selected from the group comprising alkaline metals, alkaline earth metals and ammonium. Even more preferred are inorganic salts, characterized in that X is selected from the group comprising halogen, or sulphate and in that M is selected from the group comprising alkaline metals, alkaline earth metals and ammonium. Even more preferred are inorganic salts, characterized in that when X is a halogen, M is selected from the group comprising alkaline metals, or alkaline earth metals, whereas alkaline metals are most preferred in combination with halogens. The preferred halogen is a chloride, and the preferred alkaline metals are sodium and potassium, whereas sodium is most preferred. As a result, use of sodium chloride and potassium chloride are most preferred, whereas sodium chloride is even more preferred as compared to potassium chloride. The preferable suitability of potassium and sodium chloride, preferably of sodium chloride, for the precipitation of lantibiotics, e.g. gallidermin, was a further unexpected finding, described herein. It has been shown by example 2, that use of sodium chloride results in higher yield and purity as compared to the stronger ammonium sulphate. However, alternatively ammonium salts, preferably ammonium sulphate, have also been shown to be suitable for the precipitation step as described herein.

Preferably, the precipitation with an inorganic salt is carried out at salt concentrations of at least 1.6 M or above. Preferably, the salt concentration used according to the process as described herein is about 1.6 to 10 M, more preferred about 1.6 to 5 M, even more preferred about 2.5 to 4 M, even more preferred to about 3 to 4 M. However, as described above, it is in knowledge of a person skilled in the art that the upper value of molarity is defined by the solubility of the salt at the used temperature. For example, sodium chloride is soluble in water up to 4.2 to 4.4 M at room temperature. Thus, the alkaline halogeno salts, e.g. sodium chloride, are preferably used in concentration of about 2.6 M to 4.5 M, more preferred of about 3.1 M to 4.2 M, even more preferred of about 3.2 to 3.5 M, most preferred at about 3.4 M.

According to a further embodiment of the precipitation step as described herein, the fermentation broth is adjusted to a neutral and/or slightly alkaline pH prior to the precipitation. Thus, the precipitation step is preferably performed at pH of about 7 to 10, more preferably at pH of about 7.5 to 9, even more preferably at a pH of about 7.8 to 8.8, most preferred at a pH of about 8.0. Precipitation at a slightly alkaline pH, preferably within the ranges described supra, is most preferred in combination with use of a halogen salt, preferably the alkaline halogen salt, most preferably with sodium chloride.

According to a further embodiment of the precipitation step as described herein, the salt precipitation is carry out at room temperature (about 20 to 25° C.) or less than room temperature. However, it is also in the knowledge of a person skilled in the art that the precipitation step can also preformed at temperatures slightly higher or higher than the room temperature. In general, the higher the temperature the higher the risk that the product is negatively affected. From an economic point of view, it is normally aimed to perform all the process step close to room temperature. It has surprisingly been found, that this works well with lantibiotics, preferably with gallidermin.

It has been shown for the processes described herein, that after 30 min of precipitation (under stirring), more than 80% of the product in total are salted out. The purity was about more 90% in total. Preferably the precipitation is performed for at least 30 min, preferably under stirring. Even more preferred, the precipitation is performed for at least 30 min, but stopped before the purity is decreased below about 75%, preferably below about 78%, even more preferred below about 80%, even more preferred below about 82%, even more preferred below about 84%, even more preferred below about 86%, even more preferred below about 88%, most preferred below about 90%. The product yield can be measured by standard quantitative HPLC analysis.

According to further embodiment, the precipitation is performed until at least about 70%, preferably about 75%, even more preferably until 75%, even more preferably until 80%, even more preferably until 82%, even more preferably until 85%, even more preferably until 90% in total of the product is salted out, but is stopped before the purity is decreased below about 75%, preferably below about 78%, even more preferred below about 80%, even more preferred below about 82%, even more preferred below about 84%, even more preferred below about 86%, even more preferred below about 88%, most preferred below about 90%.

According to a further embodiment, the precipitation step is performed for about 30 min to 2 h, preferably for about 30 min to 1 h, preferably, for about 30 min. Preferably, the precipitation mixture is initial stirred for at least 30 min, more preferably for 30 min. Thus, the present manufacture process for lantibiotics as described herein, comprises as an initial purification step a salt precipitation for about 1 h, wherein the first 30 min are conducted under stirring. Preferably, the salt is added over a 30 min period, with stirring, and then stirring is continued for 30 min.

It is herewith understood that the process described herein may comprise any variation described for each parameter, even if the specific combination of parameters is not explicitly mentioned. For example, the process for the manufacture of the lantibiotics as described herein also comprises an adjustment of the fermentation broth to pH 8.0 to 8.5 prior to the precipitation step, precipitation with 3 to 4 M of a halogen salt, preferably sodium chloride, at room temperature, for at least 30 min under stirring followed by another 30 min with stirring. The process described herein, also encompasses a process for the manufacture of the lantibiotics as described herein, wherein said process comprises an adjustment of the fermentation broth to pH 7.5 to 9.0 prior to precipitation, precipitation of the lantibiotic with about 3.4 M of a halogen salt, preferably sodium chloride, at room temperature until at least 80% of the lantibiotic is salted out, but wherein the precipitation step is stopped before the purity is decreased below 90% in total.

According to a further embodiment, the process for the manufacture of the lantibiotic as described herein, is characterized in that the purification process further comprises the purification step: subjecting the peptide obtained from the precipitation step, to a washing step, a single chromatographic purification step, a drying step or to crystallization.

It has been found that the purification can be further increased when the first precipitate, preferably separated by centrifugation or filtration, wherein filtration is most preferred, is washed with a solution, comprising the same salt and the same salt concentration as used for the precipitation. Optionally, the salt concentration within the wash solution can be slightly increased as compared to the precipitation solution. Preferably, the wash volume was equivalent to one tenth to half, or one tenth to three quarters of the volume of the initial product solution. After the wash step, the product containing precipitate is separated, preferably by a centrifugation or filtration step. Both, the centrifugation and filtration step are well known to a person skilled in the art. Thus, process described herein relates to a method for the manufacture of a lantibiotic peptide, comprising a fermentation and a purification step, wherein the purification step comprises the step: (i) precipitation of the lantibiotic peptide from a cell culture supernatant by adding an inorganic salt and obtaining the precipitate, (ii) washing the precipitate with a solution comprising the same salt as used for the precipitation and obtaining the precipitate. Preferably, prior to the precipitation step, cells are removed and the pH is adjusted as described supra.

According to a further embodiment of the process for the manufacture of lantibiotics, preferably gallidermin, as described herein, the precipitate obtained directly from the precipitation step or alternatively from the washing step with the same salt, both described supra, is washed in water, preferably in 1/20^{th} of the initial volume. Preferably the wash step with water is repeated. The product containing precipitate is obtained after each wash step by centrifugation or filtration, whereas filtration is most preferred. Thus the process described herein relates to a method for the manufacture of a lantibiotic peptide, comprising a fermentation and a purification step, wherein the purification step comprises the steps: (i) precipitation of the lantibiotic peptide from a cell culture supernatant by adding an inorganic salt and obtaining the precipitate, (ii) optionally washing the precipitate with a solution comprising the same salt as used for the precipitation and obtaining the precipitate, (iii) optionally washing the precipitate of steps (i) or (ii) with water and obtaining the precipitate, (iv) optionally repeating step (iii). Preferably, prior to the precipitation step, cells are removed and the pH is adjusted as described supra.

According to a further embodiment of the process for the manufacture of lantibiotics, preferably gallidermin, as described herein, the precipitate obtained directly from the precipitation step or alternatively from the washing steps, all described supra, is dissolved in a suitable buffer, preferably in an acetic buffer, even more preferred in 1% (v/v) acetic acid. Thus, the product containing precipitate (i) of the initial precipitation step, or (ii) of the wash step with the same salt, or (iii) of the first, second or any further wash step with water, is dissolved in a suitable buffer, preferably in an acetic buffer, even more preferred in about 1% to 2% (v/v) acetic acid, most preferably in about 1% acetic acid. According to a further preferred embodiments, that buffer also comprises 25 to 50% (v/v) of an alcohol, preferably ethanol. Thus a preferred acetic buffer comprises about 1 to 2% acetic acid and about 15 to 50% preferably 20 to 40% (v/v), most preferably 40% (v/v) alcohol, preferably ethanol. Subsequently, the dissolved product can be titrated to a desired pH and finally filtered and frozen, or freeze-tried or crystallized. Methods for freezing, freeze drying or crystallization are well known to a person skilled in the art, and can apply to the further processing of the lantibiotics, preferably of gallidermin. Examples of these processes are described in (Scopes, R. K., Protein Purification: Principles and Practice. Springer, 1993). Thus the process described herein relates to a method for the manufacture of a lantibiotic peptide, comprising a fermentation and a purification step, wherein the purification step comprises the step: (i) precipitation of the lantibiotic peptide from a cell culture supernatant by adding an inorganic salt and obtaining the precipitate, (ii) optionally washing the precipitate with a solution comprising the same salt as used for the precipitation, and obtaining the precipitate, (iii) optionally washing the precipitate of steps (i) or (ii) with water and obtaining the precipitate, (iv) optionally repeating step (iii), (v) dissolving the precipitate of steps (ii) to (iv) in an acetic buffer, preferably in 1% acetic acid, and (vi) titrating the dissolved product to a desired pH and finally filtered and frozen, or freeze-dried or crystallized. Preferably, prior to the precipitation step, cells are removed and the pH is adjusted as described supra.

The purity of the product could be further improved to >98 area % by preparative HPLC or LPLC, preferably by reverse phase chromatography. Methods for desalting and/or purification products using reverse phase chromatography are well known to a person skilled in the art, and described for example in (Scopes, R. K., Protein Purification: Principles and Practice. Springer, 1993). For example, suitable chromatography media that can be used are Amberchrom HPR10, XT20, cg300c and cg161c (Rohm and Haas, Philadelphia USA), and the like. Thus the process described herein relates to a method for the manufacture of a lantibiotic peptide, comprising a fermentation and a purification step, wherein the purification step comprises the step: (i) precipitation of the lantibiotic peptide from a cell culture supernatant by adding an inorganic salt and obtaining the precipitate, (ii) optionally washing the precipitate with a solution comprising the same salt as used for the precipitation, and obtaining the precipitate, (iii) optionally washing the precipitate of steps (i) or (ii) with water and obtaining the precipitate, (iv ) optionally repeating step (iii), (v) subjecting the lantibiotic peptide obtained from any of the proceeding steps (i) to (iv) to a single chromatographic purification step, preferably, wherein said chromatographic purification step is reverse phase chromatography. Preferably, prior to the precipitation step, cells are removed and the pH is adjusted as described supra.

According to a further embodiment, the precipitate obtained (i) directly from the precipitation step, (ii) from the washing step with the same salt, (iii) the first, second or any further washing step with water, as described supra, can be loaded on to reverse phase chromatography column, which is then washed with a solution in water, for example of 16% (v/v) acetonitrile, preferably together with 0.1% (v/v) trifluoroacetic acid in water, or the like. The product can then be eluted from the column with a gradient of acetonitrile, preferably from 20-40% (v/v), e.g. from 24-36% (v/v) acetonitrile in water, or the like. Preferably, the elution buffer can also include trifluoroacetic acid and the like, preferably from about 0.01 to 0.5% (v/v), even more preferably at about 0.1% (v/v). The organic solvent, is then removed from the product solution by distillation at low pressure, the product is titrated to a desired pH and finally filtered and frozen, or freeze-dried or crystallized. The collection of product from HPLC column can be adjusted to further increase the purity, however with a consequent in decrease in yield)

Alternatively, the precipitate obtained (i) directly from the precipitation step, (ii) from the washing step with the same salt, (iii) the first, second or any further washing step with water, as described supra, is suspended in an aqueous buffer, comprising ethanol and acetic acid. Preferably, the ethanol concentration is about 5 to 50% (v/v), even more preferred 20 to 45% (v/v), most preferred about 40% (v/v) and the acetic acid is of about 0,1 to 10% (v/v), preferably of about 0,5 to 5% (v/v), most preferred of about 1.0% (v/v). The resulting product solution was optionally filtered, whereas the filtration step is preferably used, and loaded on to a column for low pressure reverse phase chromatography, e.g. onto Amberchrom cg300c (Rohm and Haas, Philadelphia USA). Similar reverse phase chromatographic media would also be suitable, for example, Amberchrom cg161c, or the like. It is in the general knowledge of a person skilled in the art to select a suitable reverse phase chromatographic media for realizing the process as described herein. The column is then washed with a suitable buffer comprising for example acetonitrile and preferably trifluoroacetic acid in water, preferably about 16% acetonitrile and about 0.1% trifluoroacteic acid in water. However, any other suitable aqueous buffer, containing an organic solvent and an acid substance can be used in order to perform the reverse phase chromatography. Product elution is preferably done with a strong organic solvent, for example with about 60 to 90% (v/v) acetonitrile in water, preferably with about 80% acetonitrile in water. The organic solvent, is then removed from the product solution by distillation at low pressure, the product is titrated to a desired pH and finally filtered and frozen, or freeze-dried or crystallised.

Trifluoroacetic acid, if used during purification process, can optionally be removed from the Trifluoroacetic acid containing material by standard procedures well known in the art. For example, Trifluoroacetic acid free material can be obtained by incubation the Trifluoroacetic acid containing material with an ion-exchange matrix, preferably an anion-exchange matrix (e.g. SAX counterion hydrogencarbonate), until most of the Trifluoroacetic acid, preferably more than 90%, even more preferably more than 95%, most preferably about than 99.9% of the Trifluoroacetic acid is bound to the ion-exchange matrix. The amount of the ion-exchange matrix that is necessary depends on the binding capacity and the total content of Trifluoroacetic acid but exceeded the maximal binding capacity by minimal 10%. After at least 30 min of incubation, the solution is separated from the ion-exchanger. After washing the ion-exchange matrix with 1-2 tenths of the starting volume of a washing buffer of acetonitrile in water, that washing buffer preferably contains 10-20% acetonitrile in water, the combined solution is then distilled at low pressure to remove the acetonitrile until the solution became slightly turbid. Other well known methods to remove Trifluoroacetic acid or to exchange the Trifluoro-salt against other counterions can be used as well to produce either the free base or other salts like the hydrochloride or the acetate.

According to a further embodiment, the process described herein relates to a method for the manufacture of a lantibiotic peptide, comprising a fermentation and a purification step, wherein the purification step comprises the step: (i) precipitation of the lantibiotic peptide from a cell culture supernatant by adding an inorganic salt, and obtaining the precipitate, (ii) washing the precipitate with a solution comprising the same salt as used for the precipitation and obtaining the precipitate, (iii) subjecting the lantibiotic peptide obtained from the washing step (ii) to a single chromatographic purification step, preferably, wherein said chromatographic purification step is a reverse phase chromatography, (iv) removing the organic solvent from the product solution by distillation at low pressure, (v) titrating it to a desired pH and finally freeze-dried it. Preferably, prior to the precipitation step, cells are removed and the pH is adjusted as described supra.

### EXAMPLES

The following examples serve to further illustrate the present invention; but the same should not be construed as limiting the scope of the invention disclosed herein. Rather more, the following examples shall illustrate the general inventive concept of the process, as described herein.

### Example 1

### Fermentation of Gallidermin

Frozen culture (WCC):

One vial of Staphylococcus gallinarum (Tü 3928; DSM 4616) of frozen stock (Zellbank BO-002; Kampagne 1940122004033, Reference 35/3/18) was used to aseptically inoculate an agar plate. The agar culture was incubated at 37° C. for 1 day, followed by 1 day at 4° C. (with Parafilm—but this step is not necessary). This culture was used for inoculation of first seed. For every production a new vial must be used.

Plate Culture Medium:

| Yeast-Extract Hy-Yest 455 | 5 g/L | Kerry Bioscience |
|---|---|---|
| Glucose × H2O | 1 g/L | Cerestar or Merck |
| NaCl | 5 g/L | Fluka or Merck |
| Bacto Agar | 12 g/L | Bekton Dickinson |
| H₂O | Deion. | |
| pH | 7.3 | (before sterilization) |

Pre-Culture or First Seed (2 Shake Flasks; 500 ml):

One piece of cultured plate (ca. 0.5×0.5 cm) was transferred aseptically to two 500 ml baffled shake flasks (one baffle), containing 100 ml of sterilized seed medium. The seeded flasks are incubated on a rotary shaker (140-180 rpm) at 37° C. for 16-18 h. End product titre should be in the range of: ≧100 mg/L.

Medium:

| | | | |
|---|---|---|---|
| Yeast-Extract Ohly Kat | 50 | g/L | Deutsche Hefewerke |
| CaCl₂ × H₂O | 45 | g/L | Merck |
| Maltose × H₂O | 5 | g/L | Merck |
| PEG600/Genapol EP 0244(1:1) | 0.5 | ml/L | Fluka/Clariant |

| | | |
|---|---|---|
| H₂O | Deion. | |
| pH | 7.3 | Before sterilisation |

Second Seed (Or Laboratory Fermenter):

Second Seed Medium:

| | | | |
|---|---|---|---|
| Yeast-Extract Ohly Kat | 50 | g/L | Deutsche Hefewerke |
| CaCl₂ × H₂O | 45 | g/L | Merck |
| Maltose × H₂O | 5 | g/L | Merck |
| PEG600/Genapol EP 0244(1:1) | 0.5 | ml/L | Fluka/Clariant |

| Feeding: Glucose Monohydrat (40%, | | Cerestar |
|---|---|---|
| w/w) | | |
| H₂O | Deion. | |
| pH | 7.3 | Before sterilisation |

The first seed (2.0%) is transferred aseptically to a 20 L bioreactor with the same medium as in the pre-culture (=seed medium). The seeded jar or flask or the culture in the bioreactor is incubated at 37° C. for up to 24 h. After 5-6 hours a pO₂ shift occurs and at this time glucose feeding is started (feeding profile see under production fermentation). To prevent foam formation 0.05 ml/L of a 1:1 mixture of Genapol EP 0244 and PEG 600 is added before sterilization. If the fermenter is used as inoculum for the production fermenter then the culture is incubated only for 16-18 h and then transferred to the production bioreactor (reason: product titre should be <200 mg/L when transferred)

Production Fermentation:

The production fermentation was performed in a 1500 L tank with working volumes of 1000 L (NB up to now 20 L fermenter scale (10 L working volume, 1.5 m3 is planned). The sterilized medium is cooled down to 37° C., and inoculated with the second seed. The amount of inoculum was 0.5%. The fermentation conditions were as follows:

| | |
|---|---|
| Medium: | the same as for seed culture |
| Incubation temperature: | 37° C. |
| pH: | not controlled, but pH at inoculation should |
| | be fixed at 6.3 |
| preventing foaming: | 0.5 mL/L of a 1:1 mixture of Genapol and |
| | PEG 600 was used |
| Stirrer rate: | starting at 300 rpm |
| Aeration rate: | 0.5 vvm |

Two different feeding-profiles were tested

| | |
|---|---|
| Feeding A: | After pO₂ shift (after 5-6 h) the feeding is started with a |
| | feed rate of 6.5 kg/(m³ h) with a constant slope of 0.15 |
| | kg/(m³ h) (A). |
| Feeding B: | After 5 hours of feeding the feed-rate is increased by 1 |
| | kg/(m³) with the same slope as above (B): |

A typical fermentation profile for the production gallidermin as described above is shown in FIG. 1.

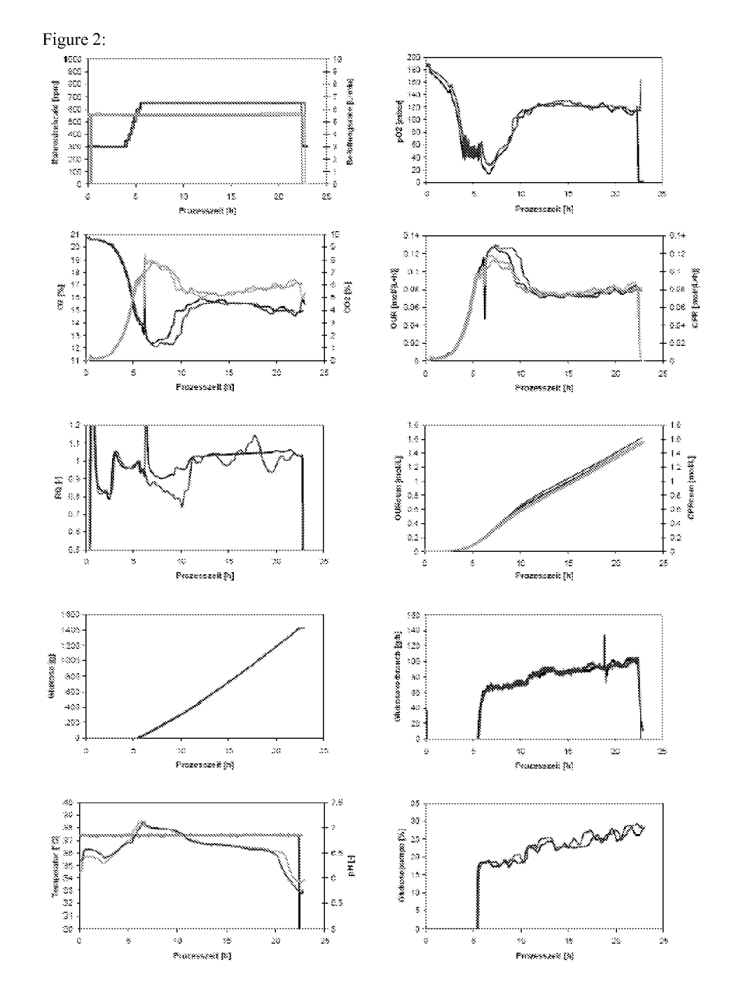

Based on all findings several fermentations were performed under the same conditions (see above) to see if the process is robust and reproducible. FIG. 2 shows the typical profiles of two standard fermentations resulting in a productivity of gallidermin as described herein.

### Example 2

### Purification of Gallidermin

Starting from the culture supernatant of the fermentation process as described under Example 1, gallidermin was purified by the following procedures:

Procedure A: Precipitation with Ammonium Sulfate

After fermentation the cells were removed by centrifugation and the product was precipitated from the cell-free supernatant (pH 6.7) by the addition, with gentle stirring over 30 min at room temperature, of 314 g/l ammonium sulphate, which is equivalent to 50% saturation.

In a separate experiment precipitation with various concentrations (saturations) of ammonium sulphate were tested.

| % saturation of ammonium sulphate | Yield (%) |
|---|---|
| 20 | 9 |
| 30 | 31 |
| 40 | 69 |
| 50 | 76 |
| 60 | 74 |

The suspension was stirred slowly for a further 30 min and then the precipitate was collected by filtration. The precipitate was dissolved in a 40% ethanol, 1% acetic acid solution in water (¼ of the starting volume). The yield of the product was 87%, and the purity, as measured by analytical HPLC was 93.6 area %.

The purity of the product could be further improved to >98 area % by preparative HPLC. The chromatography medium was Amberchrom HPR10 (XT20 was also tested successfully). The product was loaded on to the column, which was then washed with a solution in water of 16% acetonitrile, 0.1% trifluoroacetic acid. The product was eluted from the column with a gradient from 24-36% acetonitrile.

Procedure B: Precipitation with NaCl

Variant A:

1. After fermentation, the cells were removed by centrifugation and the product was precipitated from the cell free supernatant by the addition of 200 g/L NaCl with gentle stirring over 30 min at room temperature. Precipitation was continued for 30 min.

1a). In a separate experiment the effect of precipitation time on the yield and purity of the product was tested (200 g/L NaCl, pH 6.7, room temperature):

| Precipitation time | Yield | Purity after precipitation |
|---|---|---|
| (h) | (%) | (area % by HPLC) |
| 0.5 | 83.2 | 91.5 |
| 1.0 | 80.0 | 91.1 |
| 2.0 | 79.4 | 90.9 |
| 19 | 81.9 | 88.1 |

1b). In a further experiment, the effect of NaCl concentration on the product yield and purity was tested (precipitation time 30 min, pH 6.7, room temperature):

| NaCl concentration | Yield | Purity after precipitation |
|---|---|---|
| (g/L) | (%) | (area % by HPLC) |
| 310 | 86.0 | 79.6 |
| 279 | 85.7 | 81.6 |
| 248 | 88.3 | 83.3 |
| 217 | 85.6 | 85.7 |
| 186 | 81.2 | 86.3 |
| 155 | 74.4 | 86.9 |

1c). In a further experiment, the effect of the pH of the product solution before precipitation on the yield and purity of the product was tested (precipitation time 30 min, 200 g/L NaCl, room temperature):

| | Yield | Purity after precipitation |
|---|---|---|
| pH | (%) | (area % by HPLC) |
| 6.7 (direct from the | 59.8 | 92.8 |
| fermenter) | | |
| 7.5 | 75.0 | 94.2 |
| 8.0 | 83.7 | 93.9 |
| 8.5 | 90.4 | 92.8 |

2. The product precipitate was centrifuged and washed with a 200 g/L solution of NaCl. The wash volume was equivalent to half of the volume of the initial product solution.

3. The precipitate was centrifuged again and suspended in 1/20th of the initial volume of water.

4. After centrifugation, the precipitate was suspended in 1/20th of the initial volume of water and centrifuged for a second time.

5. The product precipitate was dissolved in 1% acetic acid, and could then be titrated to a desired pH with a NaOH solution, and finally filtered and frozen.

Variant B:

1. After fermentation, the cells were removed by centrifugation and the product was precipitated from the cell free supernatant by the addition of 200 g/L NaCl with gentle stirring over 30 min at room temperature. Precipitation was continued for 30 min.

2. The product precipitate was centrifuged and washed with a 200 g/L solution of NaCl. The wash volume was equivalent to half of the volume of the initial product solution.

3. The precipitate was centrifuged again and suspended in 1/20th of the initial volume of water.

4. A solution of 20% ethanol, 2% acetic acid in water was added to give a final volume of ¼ of the initial volume. A larger volume is also possible.

5. The product solution was filtered and loaded on to a column of Amberchrom cg300c (low pressure reverse phase chromatography). Similar reverse phase chromatographic media would also be suitable, for example, Amberchrom cg161c. The column was washed with 16% acetonitrile, 0.1% trifluoroacteic acid in water, and then the product was eluted with 80% acetonitrile in water.

6. Acetonitrile was removed from the product solution by distillation at low pressure.

7. The product was freeze-dried. The overall yield was 57%, the purity by HPLC was 95% and the content was 68%.

Variant C:

1. After fermentation, the cells were removed by centrifugation and the cell-free supernatant adjusted to pH 8.0 with 0.5 M NaOH. The product was precipitated from the cell-free supernatant by the addition of 200 g/L NaCl and 17 g/L Celite with gentle stirring over 30 min at room temperature. Precipitation was continued for 30 min.

2. The product precipitate was filtered and washed with three portions of a 200 g/L solution of NaCl. The total salt wash volume was equivalent to three quarters of the volume of the initial product solution.

3. The precipitate was washed with one portion of water, equivalent to 1/20th of the volume of the initial product solution.

4. The precipitate was dissolved with three portions of 40% ethanol, 1% acetic acid. The total volume was one quarter of the volume of the initial product solution.

5. The product solution was filtered and loaded on to a column of Amberchrom HPR10 reverse phase HPLC resin. Other reverse phase HPLC resins such as Amberchrom XT20, or Kromasil 100A-10-C18 would also be suitable. The column was then washed with a solution in water of 16% acetonitrile, 0.1% trifluoroacetic acid. The product was eluted from the column with a gradient from 24-36% acetonitrile in water containing 0.1% trifluoroacetic acid.

6. Acetonitrile was removed from the product solution by distillation at low pressure.

7. The product was freeze-dried. Typically, purities of >98 area % were obtained. The collection of product from the HPLC column can be adjusted to increase the purity (with a consequent decrease in yield).

Variant D:

1. After fermentation, the cells were removed by centrifugation and the cell-free supernatant adjusted to pH 8.0 with 0.5 M NaOH. The product was precipitated from the cell-free supernatant by the addition of 200 g/L NaCl and 17 g/L Celite with gentle stirring over 30 min at room temperature. Precipitation was continued for 30 min.

2. The product precipitate was filtered and washed with three portions of a 200 g/L solution of NaCl. The total salt wash volume was equivalent to three quarters of the volume of the initial product solution.

3. The precipitate was washed with one portion of water, equivalent to 1/20th of the volume of the initial product solution.

4. The precipitate was dissolved with three portions of 40% ethanol, 1% acetic acid. The total volume was one quarter of the volume of the initial product solution.

5. The product solution was filtered and loaded on to a column of Amberchrom HPR10 reverse phase HPLC resin. Other reverse phase HPLC resins such as Amberchrom XT20, or Kromasil 100A-10-C18 would also be suitable. The column was then washed with a solution in water of 16% acetonitrile, 0.1% trifluoroacetic acid (TFA). The product was eluted from the column with a gradient from 24-36% acetonitrile in water containing 0.1% trifluoroacetic acid.

6. TFA is removed by treating the fractions coming from the HPLC column directly with an ion-exchanger, preferably an anion-exchanger (e.g. SAX counterion hydrogencarbonate), once or two times until 99.9% of the TFA was bound to the ion-exchanger. The amount necessary depends on the binding capacity (total content of TFA) but exceeded the maximal binding capacity by minimal 10%. After 30 min of reaction time with the ion-exchanger, the complete suspension is poured into a large glass fritt and the solution separated from the ion-exchanger. After a wash of the ion-exchange column IEC resin with 1-2 tenths of the starting volume of a 10-20% acetonitrile in water (WFI like quality), the combined solution is then distilled at low pressure to remove the acetonitrile until the solution became slightly turbid.

Other well known methods to remove TFA or to exchange the trifluoroacetate against other counterions can be used as well to produce either the free base or other salts like the hydrochloride or the acetate.

7. The product is freeze-dried. Typically, purities of >98 area % were obtained. The collection of product from the HPLC column can be adjusted to increase the purity (with a consequent decrease in yield).

1. A method for manufacturing a lantibiotic peptide, comprising the step of:
(i) precipitating the lantibiotic peptide from a cell culture supernatant by adding an inorganic salt.

2. The method according to claim 1, further comprising the step of:
(ii) subjecting the lantibiotic peptide to a washing step, a single chromatographic purification step, a drying step, or to crystallisation.

3. The method according to claim 1, wherein the inorganic salt is of the formula Mⁿ X^{m}, wherein X is selected from the group consisting of halogen, phosphate, phosphonate, sulphate, sulphonyl, acetate, M is selected from the group comprising alkaline metals, alkaline earth metals, and ammonium, n is +1 or +2, and is −1,−2, or −3.

4. The method according to claim 1, wherein the inorganic salt is an alkali halogen.

5. The method according to claim 1, wherein the inorganic salt is sodium or potassium chloride.

6. The method according to claim 1, wherein the step of precipitating the lantibiotic peptide from a cell culture supernatant by adding an inorganic salt is carried out at a salt concentration of at least 1.6 M or above.

7. The method according to claim 1, wherein the cell culture supernatant is harvested from a cell culture medium having grown no longer than to the onset of stationary phase or is from continuous bacterial cell culture.

8. The method according to claim 7, wherein the cell culture medium comprises yeast extract.

9. The method according to claim 7, wherein the cell culture medium comprises Staphylococcus culture

10. The method according to claim 7, wherein the cell culture medium comprises S. gallinarium.

11. The method according to claim 1, wherein the lantibiotic peptide is selected from the group comprising epidermin, pep 5, or gallidermin, or variants thereof comprising at least the structural motif of Formula I: [chemical structure]
